(19) United States
(12) Patent Application Publication
(10) Pub. No.: US 2011/0118432 A1
(43) Pub. Date: May 19, 2011

(54) NATURAL OIL BASED AUTOCATALYTIC POLYOLS

(75) Inventors: Haibo Zhao, The Woodlands, TX (US); Ron Herrington, Brazoria, TX (US); Frank Rodriguez, The Woodlands, TX (US)

(73) Assignee: Huntsman Petrochemical LLC, The Woodlands, TX (US)

(21) Appl. No.: 13/003,023

(22) PCT Filed: May 21, 2009
(86) PCT No.: PCT/US2009/044776

§ 371 Date: Jan. 7, 2011

Related U.S. Application Data
(60) Provisional application No. 61/081,873, filed on Jul. 18, 2008.

(51) Int. Cl. C08G 18/32; C07C 215/10

(52) U.S. Cl. 528/85; 564/506

(57) ABSTRACT

The present invention relates to a vegetable oil based autocatalytic polyol derived from the reaction product of an epoxidized vegetable oil and an alkanolamine. The autocatalytic polyol according to the present invention may be used as a replacement for all or a portion of petroleum derived polyols generally used in the production of polyurethane materials. The polyurethane materials produced may be used in a variety of applications, such as, foam, elastomer and coating applications.

### CROSS-REFERENCE TO RELATED APPLICATION

This application claims priority to U.S. Pat. App. No. 61/081,873 filed on Jul. 18, 2008 which is incorporated herein by reference.

### STATEMENT REGARDING FEDERALLY SPONSORED RESEARCH OR DEVELOPMENT

Not applicable.

### FIELD OF THE INVENTION

The present invention described herein generally relates to polyols derived from vegetable oils and their use in foams, elastomers and coatings.

### BACKGROUND OF THE INVENTION

Polyurethane and polyurea materials, such as foams, elastomers and coatings, can be found in many applications due to their wide ranging properties and ability to be easily manufactured. For instance, the exterior and cabin interior parts of an automobile contain a number of components containing urethane foams and urea coatings such as a truck bed liner, the dashboard, the steering wheel and door panels.

The production of polyurethane and polyurea is well known in the art. Polyurethane is formed by reacting isocyanate groups with hydroxyl groups while polyurea is formed by reacting isocyanate groups with amine groups. Cross-linking agents, chain extenders, blowing agents and other additives may also be added as needed.

The most common method for producing polyurethane is via the reaction of isocyanate groups with polyol groups. Polyols used in the reaction are generally petrochemical in origin and derived from propylene or ethylene oxide and various starters such as ethylene glycol and propylene glycol. However, the use of such polyols is disadvantageous for a variety of reasons. For example, since they are ultimately derived from petroleum, they are a non-renewable resource. In addition, their production requires a great deal of energy and associated costs. Finally, their price can be unpredictable as it tends to fluctuate with the price of petroleum.

Attempts have been made to move away from the use of petroleum-derived polyols to “greener” polyols derived from vegetable and plant oil. In order to function as a polyol, hydroxyl functionality must be introduced into the vegetable oil. U.S. Pat. Nos. 2,882,249, 4,025,477, 5,266,714, 5,302,626, 6,433,125, 6,573,354 and 6,686,435 describe the introduction of hydroxyl functionality into epoxidized vegetable oil via ring opening mechanisms. Although these polyols have been shown to function in polyurethane systems, they contain secondary hydroxyl groups and therefore have a much slower reactivity than petroleum derived polyols. Attempts have been made to increase reactivity by introducing alkylene oxides into vegetable oils to form primary hydroxyl group-containing polyols as described in U.S. Pat. Publication Nos. 2003/0191274, 2006/00229375 and 2007/0282117 and U.S. Pat. No. 5,521,433. However, high costs are associated with the commercial production of these materials. U.S. Pat. Publication No. 2007/0155934 describes a process in which epoxidized soybean oil is functionalized by various amines, such as ethylene diamine, to form materials having amine and secondary hydroxyl functionality. However, such materials tend to crosslink and are also highly viscous or solid thus limiting their use in polyurethane production.

Thus, a need continues to exist for an improved vegetable oil based polyol having increased reactivity which can be used in manufacturing polyurethane materials. Also needed is a method of producing polyurethane materials based on the reaction of isocyanates, either alone or as a prepolymer, with the improved vegetable oil based polyol.

### SUMMARY OF THE INVENTION

The present invention relates to a vegetable oil based autocatalytic polyol comprising a reaction product of an epoxidized vegetable oil and an alkanolamine wherein the reaction product contains primary hydroxyl, secondary hydroxyl and tertiary amine functionality thus making the autocatalytic polyol substantially more reactive than conventional vegetable oil based polyols.

In another embodiment, the present invention relates to a method of producing the vegetable oil based autocatalytic polyol by reacting an epoxidized vegetable oil with an alkanolamine.

In yet another embodiment, the present invention relates to a process for producing a polyurethane material including reacting a polyisocyanate with the vegetable oil based autocatalytic polyol of the present invention. In a further embodiment, a method is described for producing a polyurethane material by reacting an A-side reactant containing a polyisocyanate with a B-side reactant containing a polyol component wherein the polyol component includes the vegetable oil based autocatalytic polyol and optionally a petroleum derived polyol.

The polyurethane materials produced in accordance with the present invention may be used in a variety of applications including flexible foam, rigid foam, elastomer and coating applications.

### BRIEF DESCRIPTION OF DRAWINGS

FIGS. 1 and 2 describe the reactivity of the autocatalytic polyols of the present invention as compared to petroleum derived polyols.

### DETAILED DESCRIPTION OF THE INVENTION

Various embodiments of the present disclosure generally relate to a novel vegetable oil based autocatalytic polyol that may be reacted with a polyisocyanate to form a polyurethane material. By “autocatalytic” it is meant the vegetable based polyol initiates a reaction by itself in the absence of a catalyst. In particular, certain embodiments disclosed herein relate to a vegetable oil based autocatalytic polyol comprising the reaction product of an epoxidized vegetable oil and an alkanolamine wherein the reaction product comprises primary hydroxyl, secondary hydroxyl and tertiary amine groups. In another embodiment, the reaction product contains a majority of primary hydroxyl groups. The term “vegetable oil” collectively refers to oils obtained from naturally grown, plant sources but notably from plant crops or seeds. By “majority of primary hydroxyl groups” is meant that the reaction product contains more than 50% primary hydroxyl groups based on the total amount of hydroxyl groups present. The term “reaction product” used herein may be a product or a mixture of products produced from the reaction of the epoxidized vegetable oil and alkanolamine.

Because vegetable oils are abundant, renewable, and easily processed commodities as opposed to petroleum derived polyols (which entail significant associated processing costs), the use of the vegetable oil based autocatalytic polyols according to the present invention in the production of polyurethane material may realize a significant cost savings because some or all of the more costly petroleum derived polyols may be replaced. There is also a distinct marketing advantage to marketing products that are based on environmentally friendly, renewable resources such as vegetable oils. Furthermore, because the autocatalytic polyol of the present invention, in addition to containing a majority of primary hydroxyl groups, also contains tertiary amine functionality, it is substantially more reactive than conventional vegetable oil based polyols.

Thus, in one embodiment, a vegetable oil based autocatalytic polyol of the present invention comprises a reaction product obtained from the reaction of an epoxidized vegetable oil and an alkanolamine.

The epoxidized vegetable oil suitable for use in the present invention includes those commercially available or those prepared in conventional ways. Suitable starting vegetable oil materials include, but are not limited to, palm oil, peanut oil, rapeseed oil, cottonseed oil, soybean oil, canola oil, sunflower oil, safflower oil, corn oil, olive oil, sesame oil, jathropa oil, or blends thereof. Alternatively, any partially hydrogenated vegetable oil or genetically modified vegetable oil may also be used with or in substitution of the vegetable oils listed above, and include, but are not limited to, high oleic safflower oil, high oleic soybean oil, high oleic peanut oil, high oleic sunflower oil, high erucic rapeseed oil, or blends thereof. The iodine values of these vegetable oils may range from about 40 to about 240.

The vegetable oil may be epoxidized by any conventional method including via the reaction with peracetic in the presence of an acidic catalyst or with a peroxyacid acid. Examples of peroxyacids include peroxyformic acid, peroxyacetic acid, trifluoroperoxyacetic acid, benzyloxyperoxyformic acid, 3,5-dinitroperoxybenzoic acid, m-chloroperoxybenzoic acid or any combinations of these peroxyacids. The peroxyacids may be formed in situ by reacting a hydroperoxide with the corresponding acid, such as formic or acetic acid. Examples of hydroperoxide acids include hydrogen peroxide, t-butylhydroperoxide, triphenylsilylhydroperoxide, cumylhydroperoxide or any combinations thereof. Upon epoxidation, the epoxidized vegetable oil preferably has an epoxide content of about 2 weight % to about 8 weight %. In other terms, preferably each epoxidized vegetable oil molecule contains about 2 to 6 epoxy groups.

Alkanolamines which may be reacted with the epoxidized vegetable oil in accordance with the present invention include, but are not limited to, those having the formula

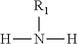

where R₁ is a linear or branched alkyl group of 1 to 10 carbons, preferably 2 to 8 carbons, and more preferably 2 to 4 carbons, that contains at least one primary hydroxyl group and

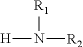

where R₁ is a linear or branched alkyl group of 1 to 10 carbons, preferably 2 to 8 carbons, and more preferably 2 to 4 carbons, that contains at least one primary hydroxyl group and R₂ is a linear or branched alkyl group of 1 to 10 carbons, preferably 2 to 8 carbons, and more preferably 2 to 4 carbons, that contains at least one primary hydroxyl group.

Examples of suitable alkanolamines include, but are not limited to, diethanolamine, ethanolamine, 2-amino-1-butanol, 2-amino-2-methyl-1-propanol, 2-amino-2-ethyl-1,3-propanediol, tris(hydroxymethyl)aminomethane, 2-amino-2-methyl-1,3-propanediol, monomethyl ethanolamine, isopropylaminoethanol, t-butylamino ethanol, ethylamino ethanol, n-butylaminoethanol, isopropanolamine, diisopropanolamine, and mixtures thereof. Preferably, the alkanolamine of the present invention is selected from the group consisting of 2-amino-1-butanol, 2-amino-2-methyl-1-propanol, 2-amino-2-ethyl-1,3-propanediol, tris(hydroxymethyl)aminomethane, 2-amino-2-methyl-1,3-propanediol, monomethyl ethanolamine, isopropylaminoethanol, t-butylaminoethanol, ethylaminoethanol, n-butylaminoethanol, isopropanolamine, diisopropanolamine, and mixtures thereof. In another embodiment, the alkanolamine is monomethyl ethanolamine. In another embodiment, the alkanolamine is diethanolamine. In yet another embodiment, the alkanolamine is monomethyl ethanolamine and ethanolamine in combination. In a still further embodiment, the alkanolamine is ethanolamine and diethanolamine in combination.

The reaction between the epoxidized vegetable oil and alkanolamine may be initiated by charging the alkanolamine and epoxidized vegetable oil to a stirred reactor to form a reactant mixture. The amount of alkanolamine charged to the reactor may range from about 50% by weight to about 70% by weight based on the total weight of the reactant mixture, and preferably from about 55% by weight to about 65% by weight based on the total weight of the reactant mixture. The amount of epoxidized vegetable oil charged to the reactor may range from about 30% by weight to about 50% by weight based on the total weight of the reactant mixture and more preferably from about 35% by weight to about 45% by weight based on the total weight of the reactant mixture. The reaction may be carried out at an elevated temperature. Suitable reaction temperatures may range from about 100° C. to about 150° C., and more preferably from about 110° C. to about 130° C. Reaction times may range from about 0.1 hours to about 12 hours, and preferably from about 2 hours to about 6 hours. Any unreacted alkanolamine and by-products produced may be removed by conventional means.

It must be emphasized that in certain embodiments, the vegetable oil based autocatalytic polyol comprises a mixture of reaction products. As shown below, according to one embodiment, monomethyl ethanolamine may be reacted with epoxidized soybean oil to form a mixture of reaction products. During the reaction, the triglyceride group is either fully cleaved from the soybean oil resulting in the formation of the reaction products and glycerin or left intact yielding chemical structures such as those described below. The reaction products contain a majority of primary hydroxyl groups based on the total amount of hydroxyl groups present, and in addition, also contain tertiary amine functionality making the autocatalytic polyol substantially more reactive than conventional vegetable oil based polyols. The mixture of reaction products is a critical element for the autocatalytic nature of the vegetable oil based autocatalytic polyol in certain embodiments.

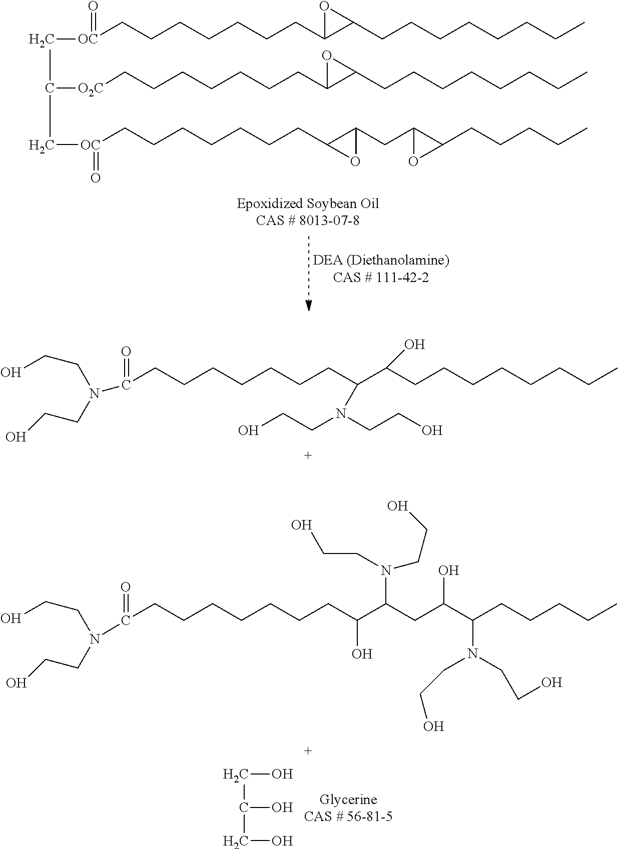

Other partial reaction products where the triglyceride is not fully cleaved to liberate glycerine include:

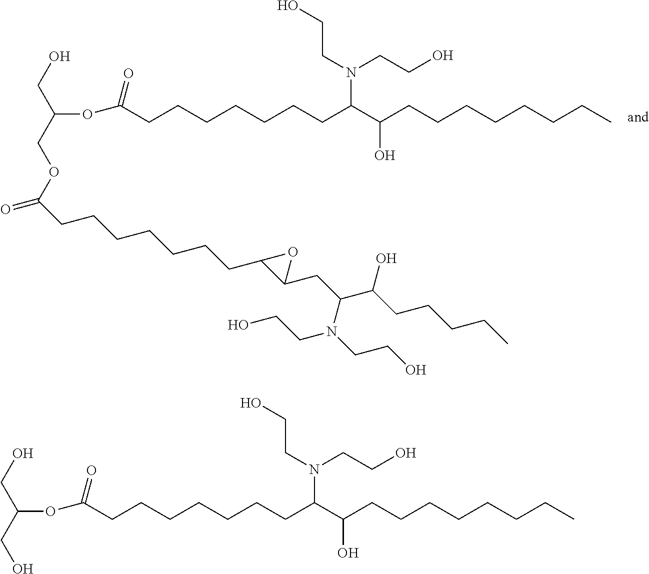

In another embodiment, the vegetable oil based autocatalytic polyol has a hydroxyl value ranging from about 400 meq KOH/g to about 600 meq KOH/g. In yet a further embodiment, the autocatalytic polyol has an amine value ranging from about 1.5 meq/g to about 3.5 meq/g.

In a still further embodiment, the vegetable oil based autocatalytic polyol produced in accordance with the present invention contains fewer odors as compared to conventional vegetable oil based polyols. Odor may be measured, for example, by using human test panels or by measuring the amount of certain odor-producing compounds that may be present in the vegetable oil based autocatalytic polyol. Examples of odor-producing compounds include lipid oxidation products, which are typically aldehyde compounds, for example, hexanal, nonanal, and decanal. In some embodiments, the vegetable oil based autocatalytic polyol of the present invention may have about 30 ppm or less hexanal, preferably, about 20 ppm or less, more preferably about 10 ppm or less, even more preferably about 5 ppm or less, most preferably about 1 ppm or less hexanal. In other embodiments, the vegetable oil based autocatalytic polyol may have about 30 ppm or less nonanal, preferably about 20 ppm or less, more preferably about 10 ppm or less, even more preferably about 10 ppm or less, most preferably about 1 ppm or less nonanal. In still other embodiments, the vegetable oil based autocatalytic polyol may have about 20 ppm or less decanal, preferably about 15 ppm or less, more preferably about 10 ppm or less, even more preferably about 5 ppm or less, most preferably about 1 ppm or less decanal. In yet other embodiments, the combined amount of hexanal, nonanal, and decanal in the vegetable oil based autocatalytic polyol may be about 80 ppm or less, preferably about 70 ppm or less, more preferably about 60 ppm or less, even more preferably about 50 ppm or less. In still another embodiment, the combined amount of hexanal, nonanal, and decanal in the vegetable oil based autocatalytic polyol may be about 40 ppm or less, preferably about 30 ppm or less, more preferably about 20 ppm or less, even more preferably about 10 ppm, most preferably about 3 ppm or less.

In accordance with another embodiment, the vegetable oil based autocatalytic polyol of the present invention may be used in the production of polyurethane materials.

Polyurethanes may be produced from the reaction of an A-side reactant with a B-side reactant. The A-side reactant may comprise a polyisocyanate while the B-side reactant may comprise the vegetable oil based autocatalytic polyol according to the present invention. The vegetable oil based autocatalytic polyol of the present invention may be used as a replacement for all of the petroleum derived polyol generally used in the production of the polyurethane material. Thus, in one embodiment, the B-side reactant includes a polyol component comprising 100% by weight of one or more vegetable oil based autocatalytic polyols.

The polyisocyanates suitable for use include unmodified polyisocyanates, modified polyisocyanates and isocyanate prepolymers. Such polyisocyanates include those represented by the formula Q(NCO)ₙ where n is a number from 2-5, preferably 2-3 and Q is an aliphatic hydrocarbon group containing 2-18 carbon atoms, a cycloaliphatic hydrocarbon group containing 5-10 carbon atoms, an araliphatic hydrocarbon group containing 8-13 carbon atoms, or an aromatic hydrocarbon group containing 6-15 carbon atoms.

Examples of suitable polyisocyanates include, but are not limited to, ethylene diisocyanate; 1,4-tetramethylene diisocyanate; 1,6-hexamethylene diisocyanate; 1,12-dodecane diisocyanate; cyclobutane-1,3-diisocyanate; cyclohexane-1,3- and -1,4-diisocyanate, and mixtures of these isomers; isophorone diisocyanate; 2,4- and 2,6-hexahydrotoluene diisocyanate and mixtures of these isomers; dicyclohexylmethane-4,4′-diisocyanate (hydrogenated MDI, or HMDI); 1,3- and 1,4-phenylene diisocyanate; 2,4- and 2,6-toluene diisocyanate and mixtures of these isomers (TDI); diphenylmethane-2,4′- and/or -4,4′-diisocyanate (MDI); naphthylene-1,5-diisocyanate; triphenylmethane-4,4′,4″-triisocyanate; polyphenyl-polymethylene-polyisocyanates of the type which may be obtained by condensing aniline with formaldehyde, followed by phosgenation (crude MDI); norbornane diisocyanates; m- and p-isocyanatophenyl sulfonylisocyanates; perchlorinated aryl polyisocyanates; modified polyisocyanates containing carbodiimide groups, urethane groups, allophnate groups, isocyanurate groups, urea groups, or biruret groups; polyisocyanates obtained by telomerization reactions; polyisocyanates containing ester groups; and polyisocyanates containing polymeric fatty acid groups. Those skilled in the art will recognize that it is also possible to use mixtures of the polyisocyanates described above.

Isocyanate-terminated prepolymers may also be employed in the preparation of the polyurethane materials. Isocyanate prepolymers may be prepared by reacting an excess of polyisocyanate or mixture thereof with a minor amount of an active-hydrogen containing compound as determined by the well known Zerewitinoff test as described by Kohler in “Journal of the American Chemical Society,” 49, 3181 (1927).

In another embodiment, the vegetable oil based autocatalytic polyol of the present invention may be used as a replacement for a portion of the petroleum derived polyol generally used. Thus, in one embodiment, the B-side reactant contains a polyol component comprising at least one vegetable oil based autocatalytic polyol and at least one petroleum derived polyol.

Examples of the petroleum derived polyol include a polyether, a polyester, a polyacetal, a polycarbonate, a polyesterether, a polyester carbonate, a polythioether, a polyamide, a polyesteramide, a polysiloxane, a polybutadiene, a polyacetone, or a mixture thereof.

Thus, in another embodiment, the B-side reactant includes a polyol component comprising from about 1% by weight to about 99% by weight of the vegetable oil based autocatalytic polyol and from about 99% by weigh to about 1% by weight of the petroleum derived polyol. In another embodiment the B-side reactant includes a polyol component comprising from about 5% by weight to about 95% by weight of the vegetable oil based autocatalytic polyol and from about 95% by weight to about 5% by weight of the petroleum derived polyol. In still another embodiment, the B-side reactant includes a polyol component comprising from about 10% by weight to about 90% by weight of the vegetable oil based autocatalytic polyol and from about 90% by weight to about 10% by weight of the petroleum derived polyol. In yet another embodiment, the B-side reactant includes a polyol component comprising from about 25% by weight to about 75% by weight of the vegetable oil based autocatalytic polyol and from about 75% by weight to about 25% by weight of the petroleum derived polyol. According to another embodiment, the B-side reactant includes a polyol component comprising from about 45% by weight to about 55% by weight of the vegetable oil based autocatalytic polyol and from about 55% by weight to about 45% by weight of the petroleum derived polyol

In addition to the polyol component, the B-side reactant may optionally comprise additives including, but not limited to: catalysts; blowing agents; crosslinking agents, flame retardants; plasticizers; internal mold release agents; surfactants; acid scavengers; water scavengers; cell regulators; pigments; dyes; UV stabilizers; plasticizers; fungistatic or bacteriostatic substances; and fillers.

Catalysts that may be used include, but are not limited to, tertiary amines such as triethylamine, tributylamine, N-methylmorpholine, N-ethylmorpholine, N,N, N′, N′-tetramethylethylenediamine, pentamethyl-diethylenetriamine, 1,4-diazabicyclo(2.2.2)octane, N-methyl-N′-dimethyl-aminoethylpiperazine, bis-(dimethylaminoalkyl)piperazines, N,N-dimethylbenzylamine, N,N-dimethylcyclohexylamine, N,N-diethyl-benzylamine, bis-(N,N-diethylaminoethyl) adipate, N,N,N′,N′-tetramethyl-1,3-butanediamine, N,N-dimethyl-.beta.-phenylethylamine, 1,2-dimethylimidazole, 2-methylimidazole, monocyclic and bicyclic amines together with bis-(dialkylamino)alkyl ethers, such as 2,2-bis-(dimethylaminoethyl)ether. However, because the vegetable oil based autocatalytic polyol of the present invention contains tertiary amine functionality, the amount of catalyst used in producing the polyurethane materials according to the present invention may be significantly reduced or preferably, not even used at all. For example, high levels of added amine catalysts are generally used in spray foam formulations containing conventional polyols so that final desired material properties may be obtained. Substituting the vegetable oil based autocatalytic polyol of the present invention into such formulations significantly reduces the levels of added amine catalyst generally needed yet final desired material properties may still be obtained.

Suitable blowing agents which may be used, include, but are not limited to, water, a hydrofluorocarbon, cyclopentane, methyl isobutyl ketone, a hydrocarbon, methylene chloride or mixtures thereof.

Suitable crosslinking agents which may be used include, but are not limited to, ethylene glycol, diethylene glycol, triethylene glycol, propylene glycol, 1,3-propanediol, 1,4-butandiol, 1,6-hexanediol, glycerol, and trimethylolpropane.

Suitable flame retardants (which, as the term is used herein, also include smoke suppressants and other known combustion modifiers), include phosphonates, phosphites, and phosphates (such as dimethyl methylphosphonate, ammonium polyphosphate, and various cyclic phosphate and phosphonate esters known in the art); halogen-containing compounds known in the art (such as brominated diphenyl ether and other brominated aromatic compounds); melamine; antimony oxides (such as antimony pentoxide and antimony trioxide); zinc compounds (such as various known zinc borates); aluminum compounds (such as alumina trihydrate); and magnesium compounds (such as magnesium hydroxide).

Internal mold release agents are compounds that may be added to assist in the removal of the polyurethane material from a mold. Suitable internal mold release agents for the present invention include those based at least in part on fatty acid esters, metal and/or amine salts of carboxylic acids, amido carboxylic acids, phosphorus-containing acids, boron-containing acids, amidines, and neutralized esters prepared from certain amine-started tetrahydroxy compounds as described in U.S. Pat. No. 5,208,268.

Surfactants (or surface-active agents) include emulsifiers and foam stabilizers, such as silicone surfactants known in the art, for example, polysiloxanes, as well as various amine salts of fatty acids, such as diethylamine oleate or diethanolamine stearate, as well as sodium salts of ricinoleic acids.

Acid scavengers are compounds that may be added to control the acidity and water concentration. Preferred acid scavengers include various orthoesters, such as trimethyl orthoformate, carbodiimides, such as 2,2′,6,6′-tetraisopropyldiphenylcarbodiimide, and epoxides, such as 3,4-epoxycyclohexylmethyl 3,4-epoxy-cyclohexylcarboxylate.

Water scavengers (or moisture scavengers) are compounds that may be added to maintain a low water content in the compositions of the present invention. Suitable water scavengers include alkali aluminosilicates.

Fillers and/or reinforcing substances, include barium sulfate, calcium carbonate, calcium silicate, clays, kieselguhr, whiting, mica, glass fibers, liquid crystal fibers, glass flakes, glass balls, microspheres, aramide fibers, and carbon fibers.

According to one embodiment, the polyurethane material may be prepared in a one-step process in which an A-side reactant is combined with a B-side reactant. The A-side may include the polyisocyanate or mixture of polyisocyanates. Different polyisocyanates may be selected to create different properties in the final product. The B-side may be a solution including at least one vegetable oil based autocatalytic polyol of the present invention or a mixture of vegetable oil based autocatalytic polyols and optionally petroleum derived polyol(s) and/or additives.

The polyurethane materials according to the present invention may be used in a variety of applications, such as, a precoat; a backing material for carpet; building composites; insulation; spray foam insulation; applications requiring use of impingement mix spray guns; urethane/urea hybrid elastomers; vehicle interior and exterior parts such as bed liners, dashboards, door panels, and steering wheels; flexible foams (such as furniture foams and vehicle component foams); integral skin foams; rigid spray foams; rigid pour-in-place foams; coatings; adhesives; sealants; filament winding; and other polyurethane composite, foams, elastomers, resins, and reaction injection molding (RIM) applications.

### EXAMPLES

### Example 1

### Synthesis of a Vegetable Oil Based Autocatalytic Polyol from Diethanolamine and Epoxidized Soybean Oil

500 grams of diethanolamine was added to a 2-L three neck flask equipped with a funnel, thermometer, stirrer, and regular condenser. The flask was then heated to 120° C. 400 grams of epoxidized soybean oil was then added to the flask through the funnel under stirring. The temperature was maintained at 120° C. and the reaction was allowed to continue for four hours. Unreacted diethanolamine and glycerin by-product were stripped from the autocatalytic polyol product at a temperature of 160° C. under full vacuum. The autocatalytic polyol product was removed and had an amine value of 2.99 meq/g and an OH value of 555 meq/g.

### Example 2

### Synthesis of a Vegetable Oil Based Autocatalytic Polyol from Monomethyl Ethanolamine and Epoxidized Soybean Oil

500 grams of monomethyl ethanolamine was added to a 2-L three neck flask equipped with a funnel, thermometer, stirrer, and regular condenser. The flask was then heated to 120° C. 400 grams of epoxidized soybean oil was then added to the flask through the funnel under stirring. The temperature was maintained at 120° C. and the reaction was allowed to continue for four hours. Unreacted diethanolamine and glycerin by-product were stripped from the autocatalytic polyol product at a temperature of 160° C. under full vacuum. The autocatalytic polyol product was removed and had an amine value of 1.94 meq/g and an OH value of 437 meq/g.

### Example 3

### Polyol Reactivity Test

The generally known Brookfield Viscosity Test (BVT) was performed on the autocatalytic polyols produced in Examples 1 and 2 and compared to petroleum derived polyols to determine the gelation rate during reaction with a polymeric MDI (Rubinate® M polymeric MDI, available from Huntsman Corporation). 100 grams of polyol and sufficient amounts of the Rubinate® M polymeric MDI were mixed together to provide a 90 index. The viscosity of the mixture was then analyzed over time. The growth in viscosity over time was used as a fingerprint of the gelation profile for the polyol given the same reaction conditions and polyisocyanate. A faster gelation per unit time indicates a more reactive the polyol. The polyols tested included:

| Polyol | Description | Type |
|---|---|---|
| JEFFSOL ® SG-360 | 4.7-functional base polyol | Petroleum based |
| | | polyether |
| JEFFSOL ® G31-28 | 3-functional, high MW | Petroleum based |
| | | polyether |
| Example 1 | Epox. Soybean oil/DEA | Vegetable oil based |
| Example 2 | Epox. Soybean oil/MMEA | Vegetable oil based |

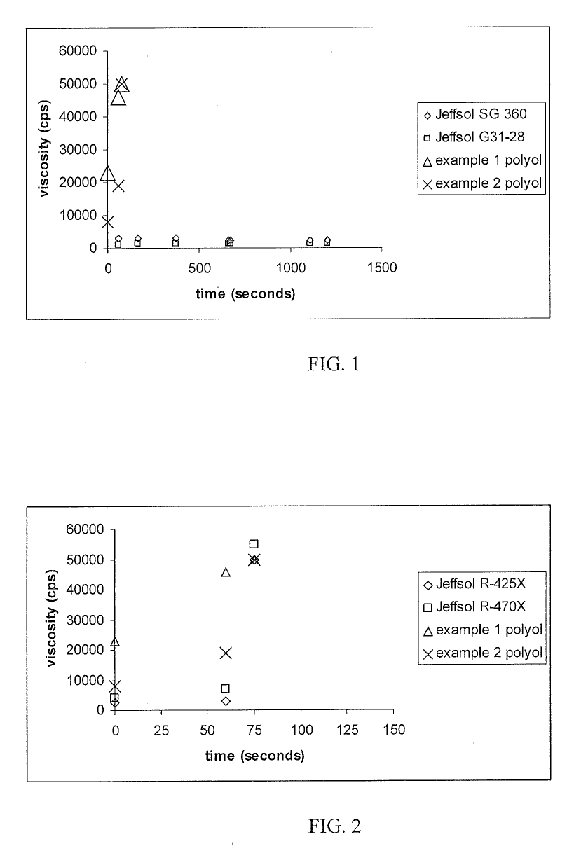

As described in FIG. 1, the vegetable oil based autocatalytic polyols of Examples 1 and 2 are “autocatalytic” in nature, and exhibited superior reactivity as compared to conventional petroleum based polyether polyols.

The BVT test was then performed as described above on Examples 1 and 2, as well as on two different petroleum derived Mannich type polyols which generally exhibit higher reactivity as compared to conventional petroleum based polyether polyols: JEFFSOL® R425X and JEFFSOL® R470X polyols (available from Huntsman Corporation). As described in FIG. 2, the vegetable oil based autocatalytic polyols of the present invention surprisingly exhibited superior reactivity as compared to the petroleum derived Mannich type polyols.

### Example 4

### Production of Polyurethane Foam

The autocatalytic polyols of the present invention were tested as a loadbearing additive in low density HR molded automotive seating type foam. A-side and B-side reactant formulations containing the following materials were prepared:

| | Example 3 | | |
|---|---|---|---|
| | Comparison | Example 4 | Example 5 |
| Component | (parts) | (parts) | (parts) |
| A-Side Reactant | | | |
| Toluene diisocyanate | 100 index | 100 index | 100 index |
| B-Side Reactant | | | |
| ¹Specflex NC-630 EO capped | 90.0 | 90.0 | 90.0 |
| polyol | | | |
| ¹Specflex NC-700 polyether polyol | 30.0 | | |
| Example 1 polyol | | 10.0 | |
| Example 2 polyol | | | 10.0 |
| ²Surface cosmetic surfactant | 0.25 | 0.5 | 0.5 |
| ³Stabilizing surfactant | 0.75 | | |
| ⁴Stabilizing surfactant | | 1.5 | 1.5 |
| ⁵Crosslinker | 1.45 | 1.45 | 1.45 |
| ⁶Gel catalyst | 0.35 | 0.35 | 0.35 |
| ⁷Blow catalyst | 0.10 | 0.10 | 0.10 |
| Water | 3.98 | 3.98 | 3.98 |

¹Petroleum derived polyols available from The Dow Chemical Co.
²DC-5169 surfactant available from Air Products Corp.
³DC-5164 surfactant available from Air Products Corp.
⁴Tegostab B-4690 surfactant available from Goldschmidt AG.
⁵Low freeze grade diethanolamine.
⁶JEFFCAT ® TD-33A catalyst available from Huntsman Corp.
⁷JEFFCAT ® ZF-10 catalyst available from Huntsman Corp.

The A-side and B-side reactants were then poured into standard 15″×15″×4″ test block molds. The foams were allowed to rise and cure for 6 minutes at a mold temperature of 150° F. After demolding, each material was mechanically crushed and further hand-crushed to ensure open cells. After one week, various physical properties of the materials were analytically tested and the results shown below.

| | Example 3 | | |
|---|---|---|---|
| Property | Comparison | Example 4 | Example 5 |
| Core density | 2.00 | 2.00 | 2.00 |
| lbs/ft³ | | | |
| 25% IFD | 29.6 | 33.1 | 37.4 |
| lbs/50 sq. in | | | |
| 65% IFD | 90 | 99.3 | 99.9 |
| lbs/50 sq. in | | | |
| 25% IFD Return | 23.7 | 26.1 | 27.5 |
| Foam Modulus | 3.05 | 3.0 | 2.67 |
| Hysteresis Loss % | 20 | 21 | 26 |
| Tensile Strength | 16.8 | 13.8 | 13.7 |
| psi | | | |
| Tear | 1.30 | 0.90 | 1.10 |
| lb/in | | | |
| Elongation % | 116 | 70 | 85 |
| 50% Reg CS % | 8 | 9 | 17 |
| 50% Wet CS % | 35 | 30 | 40 |
| Airflow | 3.6 | 3.0 | 2.7 |
| scfm | | | |
| Resiliency % | 47 | 50 | 43 |

Resiliency is one measure of comfort in an automobile seat. The resiliency of the foam produced from the autocatalytic polyol from Example 1 performed better than the comparison foam of Example 3. Furthermore, the foams produced using the vegetable oil based autocatalytic polyols of the present invention were also harder than the comparison foam.

### Example 5

### Production of Polyurethane Spray Foam

Spray foam is sprayed through a two component machine with one component being the “polyol” and the other component being the “isocyanate. The following “polyol” formulation was used:

| Component | Parts |
|---|---|
| Soy-based polyol according to this | 12.1 |
| invention | |
| Glycerine sucrose polyol GSP-280 | 10.0 |
| Terate 4020 | 48.6 |
| Tetrabromodiethyleneglycolphthalate | 5.0 |
| Dabco DC-193 | 0.8 |
| JEFFCAT ZF-20 | 0.5 |
| JEFFCAT Z-80 | 1.0 |
| Water | 2.0 |
| Blowing agent 245fa | 10.0 |

Typically, one skilled in the art balances the active hydrogens from the polyol blend with the isocyanate groups, this being called an isocyanate index. In this example, the isocyanate index of the sprayed foam was 115-125. This merely serves as an example and is not a limitation on the acceptable range of the isocyanate index.

Rate of “cure” is important in spray foam polyurethanes used in insulation which is sprayed into wall cavities. The urethane must “stick” to the wall where sprayed and not “run” to be useful in this application. To this, end a polyol that has autocatalytic behavior such as the polyol of this invention is desired since the autocatalytic nature aids in speed of “cure”. The resulting foam from the above described formulation yields the following excellent foam properties.

| Property | Value | |
|---|---|---|
| Density | 2.03 | lb/ft³ |
| K factor | 0.155 | btu-in/deg F.-sqft-hr |
| Adhesion-core | 24 | psi break stress |

The above-disclosed subject matter is to be considered illustrative, and not restrictive, and the appended claims are intended to cover all such modifications, enhancements, and other embodiments, which fall within the true scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A vegetable oil based autocatalytic polyol comprising a reaction product of an epoxidized vegetable oil and an alkanolamine and wherein the reaction product comprises primary hydroxyl, secondary hydroxyl and tertiary amine groups.

2. The autocatalytic polyol according to claim 1, wherein the epoxidized vegetable oil comprises an epoxidized soybean oil, an epoxidized palm oil, an epoxidized peanut oil, an epoxidized rapeseed oil, an epoxidized cottonseed oil, an epoxidized canola oil, an epoxidized sunflower oil, an epoxidized safflower oil, an epoxidized, corn oil, an epoxidized olive oil, an epoxidized sesame oil, an epoxidized jathropa oil or a blend thereof.

3. The autocatalytic polyol according to claim 2, wherein the alkanolamine comprises an alkanolamine of the formula 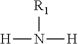
where R₁ is a linear or branched alkyl group of 1 to 10 carbons and that contains at least one primary hydroxyl group or an alkanolamine of the formula 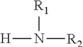
where R₁ is a linear or branched alkyl group of 1 to 10 carbons and that contains at least one primary hydroxyl group, and R₂ is a linear or branched alkyl group of 1 to 10 carbons and that contains at least one primary hydroxyl group.

4. The autocatalytic polyol according to claim 3, wherein the alkanolamine comprises diethanolamine, ethanolamine, 2-amino-1-butanol, 2-amino-2-methyl-1-propanol, 2-amino-2-ethyl-1,3-propanediol, tris(hydroxymethyl)aminomethane, 2-amino-2-methyl-1,3-propanediol, monomethyl ethanolamine, isopropylaminoethanol, t-butylamino ethanol, ethylaminoethanol, n-butylaminoethanol, isopropanolamine, diisopropanolamine, or a mixture thereof.

5. The autocatalytic polyol according to claim 3, wherein the alkanolamine is selected from the group consisting of 2-amino-1-butanol, 2-amino-2-methyl-1-propanol, 2-amino-2-ethyl-1,3-propanediol, tris(hydroxymethyl)aminomethane, 2-amino-2-methyl-1,3-propanediol, monomethyl ethanolamine, isopropylaminoethanol, t-butylamino ethanol, ethylaminoethanol, n-butylaminoethanol, isopropanolamine, diisopropanolamine, and a mixture thereof.

6. The autocatalytic polyol according to claim 4, wherein the alkanolamine comprises monomethyl ethanolamine and ethanolamine.

7. The autocatalytic polyol according to claim 4, wherein the alkanolamine comprises diethanolamine and ethanolamine.

8. The autocatalytic polyol according to claim 4, wherein the alkanolamine comprises monomethyl ethanolamine.

9. The autocatalytic polyol according to claim 1, wherein the reaction product comprises a majority of primary hydroxyl groups based on the total amount of hydroxyl groups present.

10. A method of producing a vegetable oil based autocatalytic polyol comprising reacting an epoxidized vegetable oil with an alkanolamine to form a reaction product having primary hydroxyl, secondary hydroxyl and tertiary amine groups, and removing any unreacted alkanolamine and by-products to form the vegetable oil based autocatalytic polyol.

11. The method according to claim 10, wherein the epoxidized vegetable oil is epoxidized soybean oil.

12. The method according to claim 11, wherein the alkanolamine comprises monomethyl ethanolamine.

13. The method according to claim 11, wherein the alkanolamine further comprises ethanolamine.

14. The method according to claim 11, wherein the alkanolamine comprises diethanolamine and ethanolamine.

15. A method for producing a polyurethane material comprising reacting a polyisocyanate with a vegetable oil based autocatalytic polyol wherein the autocatalytic polyol comprises the reaction product of an epoxidized vegetable oil and an alkanolamine.

16. The method according to claim 15, wherein the polyisocyanate comprises 2,4- and/or 2,6-toluene diisocyanate.

17. The method according to claim 15, wherein the polyisocyanate comprises diphenylmethane-2,4′- and/or -4,4′-diisocyanate.

18. The method according to claim 15, wherein the polyisocyanate comprises an isocyanate-terminated prepolymer.

19. A polyurethane material produced according to the method of claim 15.

20. A method for producing a polyurethane material comprising reacting an A-side reactant comprising a polyisocyanate with a B-side reactant comprising a polyol component wherein the polyol component comprises a vegetable oil based autocatalytic obtained from the reaction of an epoxidized vegetable oil and an alkanolamine.

21. The method according to claim 20, wherein the polyol component further comprises a petroleum derived polyol.

22. The method according to claim 21, wherein the polyol component comprises from about 1% by weight to about 99% by weight of the vegetable oil based autocatalytic polyol and from about 99% by weight to about 1% by weight of the petroleum derived polyol.

23. The method according to claim 22, wherein the polyol component comprises from about 10% by weight to about 90% by weight of the vegetable oil based autocatalytic polyol and from about 90% to about 10% by weight of the petroleum derived polyol.

24. The method according to claim 21, wherein the polyol component further comprises an additive.

25. A polyurethane material produced according to claim 21.
